(19) United States
(12) Patent Application Publication
(10) Pub. No.: US 2007/0172074 A1
(43) Pub. Date: Jul. 26, 2007

(54) Capacity increase in voice over packets communications systems using novel noise canceling methods and apparatus

(76) Inventor: Alon Konchitsky, Cupertino, CA (US)

Correspondence Address: STEVEN A. NIELSEN;ALLMAN & NIELSEN, P.C, 100 Larkspur Landing Circle, Suite 212, LARKSPUR, CA, 94939, US

(21) Appl. No.: 11/307,065

(22) Filed: Jan. 20, 2006

(51) Int. Cl. A61F 11/06

(52) U.S. Cl. 381/71.6; 381/71.1

(57) ABSTRACT

Improvements to the capacity of voice over packets communication systems are achieved by use of a multi-microphone system. A voice microphone collects sound waves from the user as well as unwanted background noise. A second background microphone collects ambient sound. The inputs from the two microphones retain their analog format. The signal from the background microphone is subtracted from the input of the voice microphone. The resulting signal has an increased signal to noise ratio and thus increases the capacity of a voice over packets network.

### BACKGROUND OF THE INVENTION

(1) Field of the Invention

A novel technique to cancel noise in voice over IP systems is presented. The present invention provides an increased SNR and thus dramatic increases in the realized channel capacity or bandwidth of a wire line channel as well as wireless network or cellular mobile communication systems. The capacity or “C” of a channel is often expressed in the Shannon theorem as:
C=BW×log₂(1+SNR) where
C is the channel capacity expressed in bits per second inclusive of error correction;
BW is the bandwidth of the channel expressed in hertz; and
SNR is the signal-to-noise ratio of the communication signal to the noise.

The present invention relates to voice over packets communication systems suitable for use in cell phones, radio telephones, cordless telephones, PDAs, laptop computers and in other wire line or wireless mobile devices or environments where noise reduction or cancellation is desired.

(2) The Related Art

Other two microphone noise reduction or cancellation systems are known in the related art. U.S. Pat. No. 6,415,034 (the “Hietanen patent”) describes a second background noise microphone located within an earphone unit or behind an ear capsule. Digital signal processing is used to create a noise canceling signal which enters the speech microphone. Unfortunately, the effectiveness of the method disclosed in the Hietanen patent is compromised by acoustical leakage, that is where ambient or environmental noise leaks past the ear capsule and into the speech microphone. The Hietanen patent also relies upon expensive digital circuitry.

U.S. Pat. No. 5,969,838 (the “Paritsky patent”) discloses a noise reduction system utilizing two fiber optic microphones that are placed side-by-side to one another. Unfortunately, the Paritsky patent discloses a system using light guides and other relatively expensive and/or fragile components not suitable for the rigors of cell phones and other mobile devices. Neither Paritsky nor Hietanen address the need to increase capacity in voice over packets communication systems.

Therefore, there is a need in the art for a method of noise reduction or cancellation that is robust, suitable for mobile use, and inexpensive to manufacture. The increased traffic in voice over packets communication systems has created a need in the art for means to increase signal to noise ratios in communication devices.

### BRIEF SUMMARY OF THE INVENTION

The present invention overcomes shortfalls in the related art by using two standard microphones that are positioned on a device to collect primarily either voice input and/or background noise. The background microphone is fully exposed to the environment and does not need to be concealed or otherwise protected. The two standard microphones are analog, rugged and inexpensive to manufacture. A robust and inexpensive analog sum circuit subtracts the background noise from the voice input which yields a clearer voice signal and a higher signal to noise ratio. The present invention facilitates a higher signal to noise ratio and thus increases the capacity of voice over packets communication systems.

### BRIEF DESCRIPTION OF THE DRAWINGS

FIG. 1 is a front view of a phone constructed in accordance with the disclosed invention.

FIG. 2 is a side view of a phone constructed in accordance with the disclosed invention with two microphones on the side of the phone.

FIG. 3 is a side view of a phone constructed in accordance with the disclosed invention with the voice microphone on the front of the phone and the background microphone on the back of the phone.

FIG. 4 is a block diagram of an analog sum circuit connected to the speech microphone and background microphone.

FIG. 5 is a block diagram of up to 180 degree constant or dynamic phase inverter connected to the background microphone signal.

FIG. 6 is a graph illustrating the increased capacity of a channel as a function of increased SNR.

### DETAILED DESCRIPTION OF THE INVENTION

Voice over Packets, or Voice over IP (Internet Protocol)

Voice over Internet Protocol (also called VolP, IP Telephony, Internet telephony, and sometimes Digital IP Phone) is the routing of voice conversations over the Internet or any other IP-based network. The voice data flows over a general-purpose packet-switched network, instead of traditional dedicated, circuit-switched voice transmission lines.

Protocols used to carry voice signals over the IP network are commonly referred to as Voice over IP or VoIP protocols.

Voice over IP traffic might be deployed on any IP network, including ones lacking a connection to the rest of the Internet, for instance on a private building-wide LAN.

A cellular network is a radio network made up of a number of radio cells (or just cells) each served by a fixed transmitter, normally known as a (base station). These cells are used to cover different areas in order to provide radio coverage over a wider area than the area of one cell. Cellular networks are inherently asymmetric with a set of fixed main transceivers each serving a cell and a set of distributed (generally, but not always, mobile) transceivers which provide services to the network's users.

The primary requirement for a network in cellular concept is a way for the distributed stations to distinguish the signal from its own transmitter from the signal from other transmitters. There are two common solutions to this, frequency division multiple access (FDMA) and code division multiple access (CDMA). FDMA works by using a different frequency for each neighboring cell. By tuning to the frequency of a chosen cell the distributed stations can avoid the signal from other neighbors. The principle of CDMA is more complex, but achieves the same result; the distributed transceivers can select one cell and listen to it. Other available methods of multiplexing such as Polarization division multiple access (PDMA) and time division multiple access (TDMA) cannot be used to separate signals from one cell to the next since the effects of both vary with position and this would make signal separation practically impossible. Time division multiple access, however, is used in combination with either FDMA or CDMA in a number of systems to give multiple channels within the coverage area of a single cell.

In the case of a typical taxi company, each radio has a knob. The knob acts as a channel selector and allows the radio to tune to different frequencies. As the drivers move around, they change from channel to channel. The drivers know which frequency covers approximately what area, when they don't get a signal from the transmitter, they also try other channels until they find one which works. The taxi drivers only speak one at a time, as invited by the operator (in a sense TDMA).

Increase Capacity

The wireless world comprises the following, but not limited schemes: time based and code based. In the cellular mobile environment these techniques are named under TDMA which comprises but not limited to the following standards GSM, GPRS, EDGE, IS-136, PDC, etc; and CDMA which comprises but not limited to the following standards: CDMA one, IS-95A, IS-95B, CDMA 2000, CDMA 1×EvDv, CDMA 1×EvDo, WCDMA, UMTS, TD-CDMA, TD-SCDMA, OFDM, WiMax, WiFi, etc).

For the code based standards, as the number of CDMA subscribers grows and average minutes per month increase, more and more mobile calls originate and terminate in noisy environments. The background noise does more than degrade voice quality; it also impacts network capacity.

CDMA's Rate Determination Algorithm (RDA) is designed to select Rate 1 (9.6 kbps) for speech and Rate ⅛ (1.2 kbps) for non-speech. Unfortunately, impairments such as background noise are often misinterpreted by the RDA as voice, consuming unnecessary network bandwidth.

The present invention discloses a dual microphone innovation to reduce or cancel the noise from entering the RDA, reducing the average forward-link data rate generated by the voce coder by an average of 20% in noisy conditions.

For the time based schemes, like GSM or GPRS or Edge case, improving the end-user voice signal to noise ratio, improves the listening experience of existing TDMA (time division multiple access) based networks, by the speech quality employing background noise reduction or canceling.

Although all TDMA based network, i.e. GSM voice coders perform reasonably well under optimal network conditions, the performance of Half Rate voice calls quickly deteriorates in low capacity network conditions, especially when ambient background noise is present. As a result, Half Rate calls reduce call holding time and customer satisfaction, making carriers reluctant to enable Half Rate in their networks.

The present invention removes ambient impairments and improves Half Rate calls to a voice quality level equivalent to Full Rate.

By extending the area where calls with acceptable voice quality can be made, the disclosed invention is particularly effective at cell-edges with low signal conditions.

### THE PREFERRED EMBODIMENT

In a voice over packets channel, the existence of noise is the most limiting factor to the channel capacity. FIG. 6 demonstrates the relationship between SNR and channel capacity limit as presented by Claude Shannon.

The present invention is directed toward the design and construction of a two (or more) microphone system that yields an increased SNR. A background microphone captures ambient sound or noise which is subtracted from the sound captured from the voice signal microphone. The resulting input has an increased SNR as compared to the typical single microphone system. In the single microphone system, both background noise and the desired voice single enter the communication system. In the present invention, the background noise entering the voice single microphone is removed by subtracting analogous background noise captured by the separate background microphone.

The present invention contemplates a myriad of multi-microphone configurations such as the two microphone scheme shown on FIG. 1. A phone 100 may be a cell phone or other communication device. In FIG. 1 the phone 100 has voice microphone 102 and the front and a background microphone 101 also on the front side. For reference, the phone 100 has a display 103, keypad 104, and ear speaker 105. FIG. 1 shows the best mode known to date.

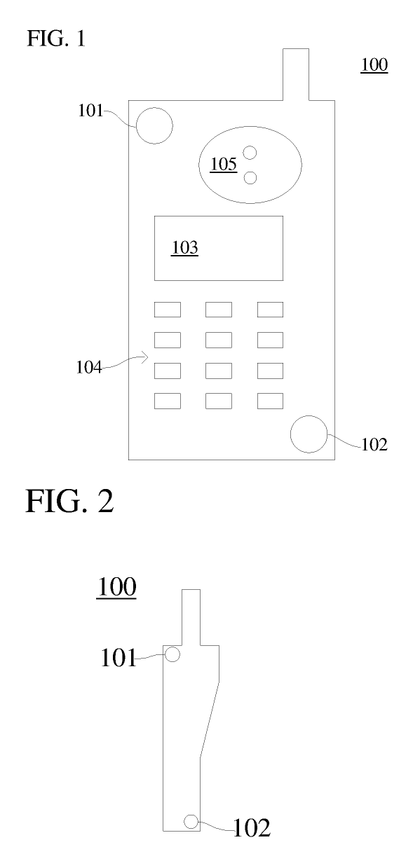

FIG. 2 shows one of the many alternative embodiments with a side view of a typical communication device 100 wherein the voice signal microphone 102 and background signal microphone 101 are located on the side of the phone.

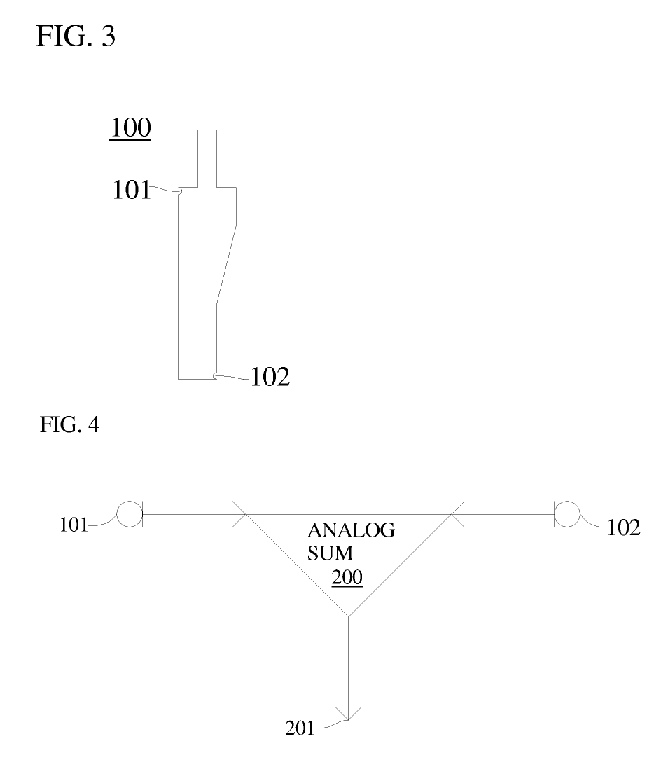

FIG. 3 shows one of the many alternative embodiments with a voice signal microphone 102 on the front of the phone 100 and a background microphone 101 placed on the back side of the phone. FIG. 3 is a side view of the phone.

FIG. 4 is a block diagram of background microphone 101 entering Sum Circuit 200 and voice signal microphone 102 also entering the Sum Circuit. The Sum Circuit creates an output at 201 wherein the background input of 101 is removed from the voice signal input of 102.

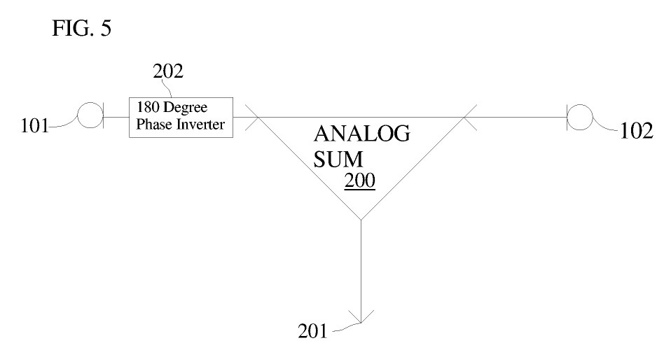

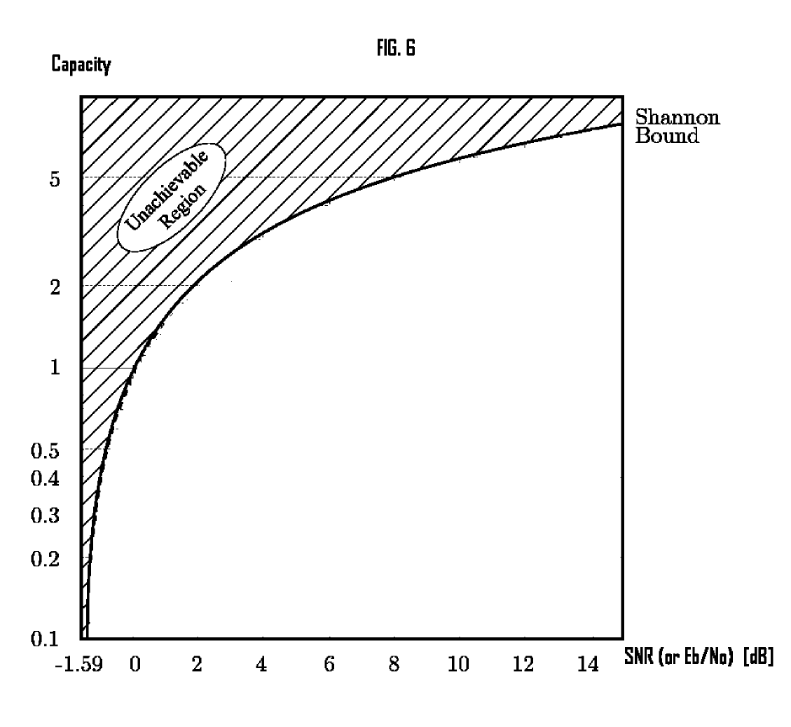

FIG. 5 is a block diagram of background microphone 101 entering a up to 180 degree constant or dynamic Phase Inverter 202 after which the inverted signal enters the Sum Circuit 200. Voice signal input 102 enters the other input to Sum Circuit 200. The increased SNR output exits the Sum Circuit at output 202.

FIG. 6 is a graph showing the relationship between increasing SNR on the horizontal x axis and increasing signal channel capacity on the vertical y axis. Due to the increased traffic over the Internet and in private networks, the increase in signal channel capacity is especially useful.

What is claimed is:

1. A method of noise reduction and/or cancellation in voice over packets systems comprising the steps of:
(a) detecting a speech signal from a microphone;
(b) detecting background noise from a microphone; and
(c) subtracting the background noise from the speech signal.

2. The method of claim 1 wherein an analog sum circuit is used to subtract the background noise from the speech signal.

3. The method of claim 1 wherein the background noise signal constantly or dynamically is inverted or phase shifted.

4. The method of claim 1 wherein the speech signal is detected by a microphone located relatively close to the speaker's mouth and background noise is detected by a microphone located relatively further from the mouth of the speaker.

5. A noise reducing and/or noise cancelling device comprising:
(a) means for detecting background noise;
(b) means for detecting a speech signal; and
(c) means for subtracting the background noise from the speech signal.

6. The device of claim 5 wherein an analog sum circuit is used to subtract the background noise from the speech signal.

7. The device of claim 5 wherein the background noise signal is up to 180 degrees inverted.

8. The device of claim 7 wherein the inverted background signal enters a sum circuit with the speech signal.

9. A method of increasing the capacity of a voice over packets channel by noise reduction or cancellation, comprising the steps of:
(a) detecting a speech signal from a microphone;
(b) detecting background noise from a microphone; and
(c) subtracting the background noise from the speech signal.

10. The method of claim 9 wherein an analog sum circuit is used to subtract the background noise from the speech signal.

11. The method of claim 9 wherein the background noise signal is inverted.

12. The method of claim 9 wherein the speech signal is detected by a microphone located relatively close to the speaker's mouth and background noise is detected by a microphone located relatively further from the mouth of the speaker.
